(19) United States
(12) Patent Application Publication
(10) Pub. No.: US 2022/0240797 A1
(43) Pub. Date: Aug. 4, 2022

(54) METHOD AND SYSTEM FOR HEMODYNAMIC MONITORING

(71) Applicant: UNIVERSITEIT GENT, Gent (BE)

(72) Inventors: Piet WYFFELS, Lichtervelde (BE); Patrick WOUTERS, Rotselaar (BE)

(21) Appl. No.: 17/612,914

(22) PCT Filed: May 29, 2020
(86) PCT No.: PCT/EP2020/065034

§ 371 Date: Nov. 19, 2021

(30) Foreign Application Priority Data
May 29, 2019 (EP) 19177472.8

(51) Int. Cl. A61B 5/02; A61B 5/0205; A61B 5/021; A61B 5/08; A61B 5/352; A61B 5/00; A61B 5/053; G16H 50/20; G16H 40/63

(52) U.S. Cl. CPC A61B 5/02028; A61B 5/0205; A61B 5/021; A61B 5/0816; G16H 40/63; A61B 5/7275; A61B 5/053; G16H 50/20; A61B 5/352

(57) ABSTRACT

A method for predicting a dynamic filling parameter for a heart-vessel system of a patient includes receiving electrocardiogram data for the patient over time, receiving data related to a respiratory cycle of the patient over time, and receiving continuous blood pressure data respectively continuous stroke volume data for the patient over time. The method also involves correlating the electrocardiogram data related to the respiratory cycle and the continuous blood pressure data respectively continuous stroke volume data thereby expressing the pulse pressure respectively stroke volume as a deconvolution of at least a function of the electrocardiogram data and a function of the respiratory cycle data. The method includes determining a value for a dynamic filling parameter representative for the hemodynamics of the patient based on the expression for the pulse pressure respectively stroke volume.

### FIELD OF THE INVENTION

The present invention relates to hemodynamic monitoring. More particularly, the present invention relates to hemodynamic monitoring of critically ill patients or patients under general anaesthesia.

### BACKGROUND OF THE INVENTION

Critically ill patients and patients under general anaesthesia need to be hemodynamically monitored. During an operation one of the tasks of an anaesthetist is the hemodynamic optimization of a patient. This means for example that he/she needs to correct for blood loss and fluid deficits.

After a few decades of research looking into fluid responsiveness, it is acknowledged that when applied correctly, dynamic filling parameters have a good capability to predict the effect of fluid loading on cardiac output. Dynamic filling parameters like Stroke Volume Variation (SVV) and Pulse Pressure Variation (PPV), thus have obtained a central place in perioperative fluid and hemodynamic management, because of their superiority in predicting fluid responsiveness. Currently, PPV is calculated as the percent change in individual pulse pressures during a ventilation cycle. National and international guidelines advise on perioperative use of these parameters for goal-directed treatment and they form the backbone of closed loop hemodynamic systems that are being developed.

The most important prerequisites to use for example PPV currently are closed chest conditions, the absence of spontaneous breathing and thus full mechanical ventilation using high tidal volumes such as for example at least 8 ml/kg, a heart rate/mechanical ventilation ratio of 3.6 and a regular heart rhythm. These limitations undermine the applicability of these parameters; especially in the ICU, and to a lesser extent in the operating theatre. The PPV parameter loses its predictive capacities when a patient has an irregular heartbeat. Applying the classic formula in AF patients typically overestimates the ventilation induced changes in pulse pressure (PP), because it cannot distinguish between the intrinsic beat to beat variation in PP based on the irregularity of the heart rhythm on the one hand and the cyclic change imposed by the ventilator on the other hand.

Some methods to determine fluid responsiveness in patients with a related condition are known.

The first is a method described by Cannesson et al. in Critical Care Medicine, 40(1), 193-198. They developed a method applied to dogs with extra-systoles (extra beats on top of a baseline regular heart rhythm) induced with a pacemaker, whereby, after exclusion of these extra beats, the traditional formula was applied to the remaining regular beats. The results of this animal's study showed good prediction capacities. After excluding extrasystoles along with the following beat and after extrapolation based on the remaining beats, their corrected SVV performed markedly better in predicting fluid responsiveness than the uncorrected SVV (ROC 0.892 vs 0.596).

However, the model is for example not applicable to patients with atrial fibrillation, because in this condition all beats are irregular, and as a result all should be excluded for analysis.

A second method is described by Vistissen et al. in international patent application PCT/DK2014/050094. They used a population with extra-systoles to determine fluid responsiveness. They use the impact of this extra-systolic beat on blood pressure for determining a dynamic filling parameter. Their concept is based on the idea to use the prolonged extra systolic filling time, as a preload changing technique. The method does not allow continuously measuring and quantifying a dynamic filling parameter and if the incidence of these extra systoles is low or absent, the variable can't be determined.

In Am J Physiol-Heart C., American Physiological Society, (2016) 310, Wyffels et al. have demonstrated a method to predict the effect of an irregular heart rhythm on the beat-to-beat variation in pulse pressure in these patients. This is based on the analysis of the duration of the 2 preceding RR-intervals of each individual heartbeat. Nevertheless, the model is not able to provide a reliable quantitative assessment of the impact of a respiratory cycle, e.g. a cycle induced by mechanical ventilation. Therefore, whereas the model allows to predict the effect of an irregular heart rhythm, it does not disclose a method for evaluating patients that are subject to mechanical ventilation, neither does it hint how to take this into account.

Another technique that has been used to predict the impact of fluid loading on cardiac output is the ‘passive leg raising’ test. By measuring the impact of the small fluid shift imposed by elevating the legs of a patient in a standard way, it is possible to predict the impact of real fluid loading. The passive leg-raising (PLR) test has the theoretical advantage that it is a ventilator independent technique with minor impact of the heart rhythm. A recent meta-analysis, that pooled the data of 23 clinical trials failed to conclude on the ability of PLR to predict fluid responsiveness in AF, because the majority of the included patients had sinus rhythm, as described in Cherpanath et al., Crit. Care Med. 44 (2016) p 981-991. In Journal of Anaesthesia, Oxford University Press 116 (2016) p 350-356,

Kim et al studied the capability of 2 techniques to predict fluid responsiveness in a group of 43 patients with AF. The first technique, PEEP induced changes in CVP failed to discriminate between responders and non-responders after a fluid bolus of 300 ml of colloids. PLR, on the contrary had some predictive abilities. A raise of 7.3% in SVI after PLR had a sensitivity of 71% and specificity of 79% to predict a Cardiac Output raise of 10%. Their reported discriminatory power (ROC of 0.771) is lower than that reported for patients in sinus rhythm however. One explanation for this result could be that the cardiac output measurements, especially the smaller ones after PLR are less reliably measured due to AF. On top of this, PLR is very unpractical to perform with on-going surgery, which undermines its widespread use in the operating theatre. The technique thus is not widely-spread in operating rooms since it does not allow continuously measuring, it requires a fast-acting cardiac measuring device and it is not always possible or convenient to change the position of the patient with ongoing surgery.

There is still room for improvement in methods and systems for determining a dynamic filling parameter.

### SUMMARY OF THE INVENTION

It is an object of embodiments of the present invention to provide good methods and systems for determining a dynamic filling parameter.

It is an advantage of embodiments of the present invention that the methods and systems are applicable to a growing population consisting of aging and more vulnerable patients, which typically suffer more from irregular heartbeats.

It is an advantage of embodiments of the present invention to accurately quantify a respiratory induced pulse pressure variation (RPPV). The respiratory induced pulse pressure variation may comprise or correspond to a ventilation induced pulse pressure variation (VPPV), i.e. a variation induced by a mechanical ventilation. Alternatively, the respiratory induced pulse pressure variation may according to embodiments of the present invention be a spontaneous breathing induced pulse pressure variation, i.e. a variation induced by a spontaneous breathing of the patient. It is an advantage of embodiments of the present invention that the obtained parameter has the potential to serve as a dynamic filling parameter for fluid responsiveness. As a further alternative, embodiments of the present invention also may allow to accurately quantify a respiratory induced stroke volume variation (RSVV), which may comprise or correspond to a ventilation induced stroke volume variation (VSVV) or which may be a spontaneous breathing induced stroke volume variation.

It is an advantage of embodiments of the present invention that it allows to incorporate not only irregular heartbeat, but also the influence of ventilation and/or spontaneous breathing and trending over time.

It is an advantage of embodiments of the present invention that the model allows for quantification of other potential influencing factors on PP changes, such as for example ventilation, loading conditions, and trending over time, influencing beat to beat changes of Pulse Pressure (PP).

The object is obtained by a system and/or method according to the present invention.

The present invention relates to a computer-implemented method for predicting a dynamic filling parameter for the heart-vessel system of a patient, the method comprising

receiving electrocardiogram data for a patient over time,
receiving data related to the respiratory cycle of the patient over time,
receiving continuous blood pressure data respectively continuous stroke volume data for a patient over time,
said electrocardiogram data, said data related to the respiratory cycle and said continuous blood pressure data respectively continuous stroke volume data being corresponding data regarding the patient recorded during a same moment in time,
the method further comprising
correlating said electrocardiogram data, said data related to the respiratory cycle and said continuous blood pressure data respectively continuous stroke volume data thereby expressing the pulse pressure respectively stroke volume as a deconvolution of at least a function of said electrocardiogram data and a function of said respiratory cycle data, and
determining a value for a dynamic filling parameter representative for the hemodynamics of the patient based on the expression for the pulse pressure respectively stroke volume.

Where in embodiments according to the present invention reference is made to correlating electrocardiogram data, data related to the respiratory cycle and continuous blood pressure data respectively continuous stroke volume data, reference is thus made to combining this data so that the data complies with an expression expressing the pulse pressure respectively stroke volume as a deconvolution of at least a function of the electrocardiogram data and a function of the respiratory cycle data. It is an advantage of embodiments according to the present invention that methods and systems are provided for accurately determining a dynamic filling parameter such as for example the respiratory induced pulse pressure variation or the respiratory induced stroke volume variation, even for patients with an irregular heartbeat, e.g. atrial fibrillation. It is an advantage that such embodiments allow accurate determination of a dynamic filling parameter for aged and vulnerable patients since these suffer more often from an irregular heartbeat. It thus is an advantage of embodiments of the present invention that arrhythmic conditions are taken into account when determining dynamic filling parameters so that these parameters can be determined for patients suffering from arrhythmia. The latter can advantageously be performed by expressing the pulse pressure and/or stroke volume as a deconvolution in at least a function of the electrocardiogram data and a function of the respiratory cycle data. Where in embodiments of the present invention reference is made to a deconvolution the latter may refer to a combination of the different functions of the data described, i.e. corresponding with an additive model whereby all influencing data are added in separate functions. Alternatively, in addition or as replacement also components or functions expressing interaction between the data may be present in the deconvolution. In some embodiments the electrocardiogram data may for example be expressed as deconvolution of functions of the RR−1 signal separately and the RR0 signal separately.

In some embodiments, the deconvolution may be a deconvolution in one or more functions of particular electrocardiogram data. In some embodiments, the deconvolution may be a deconvolution in one or more functions of data related to RR−1 and RR0. In some models, the functions may be combined in a general additive model (GAM).

It is an advantage of embodiments of the present invention that the methods and systems according to the present invention also can be applied to patients having a regular heartbeat, such that no variation is to be applied depending on the type of patient that is monitored.

It is an advantage of embodiments according to the present invention that the method takes into account irregular heartbeats for determining an accurate prediction of a dynamic filling parameter, rather than excluding it.

Expressing the pulse pressure respectively stroke volume as a deconvolution of at least a function of said electrocardiogram data and a function of said respiratory cycle data may comprise applying a gam (general additive model) model for the pulse pressure respectively stroke volume as function of at least said electrocardiogram data and said respiratory cycle data. It is an advantage of embodiments according to the present invention that accurate determination of a dynamic filling parameter can be performed during imposed ventilation. It is an advantage of embodiments according to the present invention that a determination of an accurate dynamic filling parameter can be performed continuously. It is an advantage that the determination of an accurate dynamic filling parameter can be performed without the need for further manoeuvres such as tilting the legs.

The dynamic filling parameter representative for the hemodynamics of the patient may be an expression for the respiratory induced variation of the pulse pressure or stroke volume.

Expressing the pulse pressure or stroke volume as a deconvolution of at least a function of said electrocardiogram data may comprise expressing the pulse pressure or stroke volume as a deconvolution of at least a function of the duration of a preceding RR interval in an ECG wave, wherein the RR intervals are calculated for every individual heartbeat considered. It is an advantage of embodiments of the present invention that accurate determination of a dynamic filling parameter can be performed using input of conventional data such as for example ECG data which are commonly available or can be easily obtained.

Expressing the pulse pressure or stroke volume as a deconvolution of at least a function of said electrocardiogram data may comprise expressing the pulse pressure or stroke volume as a deconvolution of at least a function of the duration of the most recent RR interval (RR₀) and a function of the duration of the RR interval (RR₋₁) preceding the most recent RR interval, whereby the RR intervals are calculated for every individual heartbeat considered.

The function of the duration of a preceding RR interval may be a spline. The spline may be a penalized cubic regression spline.

Expressing the pulse pressure or stroke volume as a deconvolution of at least a function of the respiratory cycle data may comprise expressing the pulse pressure or stroke volume as a deconvolution of at least a function of the timing of each heart beat with the respiratory cycle. It is an advantage of embodiments according to the present invention that good methods and systems are provided for determining a dynamic filling parameter such as for example the variation in pulse pressure, both for patients that are breathing spontaneously as for patients that are ventilated.

Said function of the timing of each heart beat with the respiratory cycle may be a spline. The spline may be a cyclic cubic spline.

The pulse pressure or stroke volume may be expressed as a deconvolution of at least a function of said electrocardiogram data, a function of said breathing data, and an additional function expressing a slow variation of the pulse pressure. Where in embodiments of the present invention reference is made to slow variation, the frequency of variation may be at least twice time lower than the frequency of the variation induced by the respiratory cycle. Examples of such slow variating phenomena may be Mayer waves.

The respiratory cycle data may be ventilation data. It is an advantage of embodiments of the present invention that use can be made of ventilation data that are commonly available in commercial ventilation systems.

The method may be implemented as a computer program software.

The present invention also relates to a system for predicting a dynamic filling parameter for the heart-vessel system of a patient, the system comprising

an electrocardiogram data receiving means for receiving electrocardiogram data for a patient over time
a respiratory cycle data receiving means for receiving data related to the respiratory cycle of the patient over time,
a continuous blood pressure data receiving means for receiving continuous blood pressure data for a patient over time or a continuous stroke volume data receiving means for receiving continuous stroke volume data for a patient over time,
said electrocardiogram data, said data related to the respiratory cycle and said continuous blood pressure data respectively continuous stroke volume data being corresponding data regarding the patient recorded during a same moment in time.

The system also comprises a processor being configured for correlating said electrocardiogram data, said data related to the respiratory cycle and said continuous blood pressure data respectively continuous stroke volume data thereby expressing the pulse pressure respectively stroke volume as a deconvolution of at least a function of electrocardiogram parameters and a function of respiratory cycle parameters, and for determining a value for a dynamic filling parameter representative for the hemodynamics of the patient based on the expression for the pulse pressure respectively stroke volume.

The system furthermore may be programmed for performing a method as described above.

The electrocardiogram data receiving means may be an ECG monitor.

The respiratory cycle data receiving means may be a ventilator. According to some embodiments, the respiratory cycle data also can be obtained from monitors or from bio-impedance measurements.

Particular and preferred aspects of the invention are set out in the accompanying independent and dependent claims. Features from the dependent claims may be combined with features of the independent claims and with features of other dependent claims as appropriate and not merely as explicitly set out in the claims.

These and other aspects of the invention will be apparent from and elucidated with reference to the embodiment(s) described hereinafter.

### BRIEF DESCRIPTION OF THE DRAWINGS

FIG. 1 illustrates the terminology and schematic representation of the analysis of the raw data as used in embodiments of the present invention. FIG. 1 in panel A and B show raw data of a 60 s observation period. The continuous pulse pressure (the upper line in the graph)) and the ECG signal (lower line in the graph) of the consecutive beats are shown. Line 3 shows the timing of the ventilator cycles (VC). For each pulse (pi) the pulse pressure (PP) and 4 variables were extracted. The 2 preceding RR intervals (RR_{0,i} and RR_{-1,i}), the relative timing within each VC (line 3) and its timestamp (line 4) are shown. This procedure is repeated for every pulse within the 60 s input window.

FIG. 2 illustrates a schematic presentation of the analysis procedure according to embodiments of the present invention. The upper panel shows the input for an example of a full 60 s window. All consecutive, time stamped beats are plotted against the individual PP (mmHg). All individual beats are coded according to the procedure described in FIG. 1. The middle panel shows the modelling, wherein a general additive model is calculated. PP is predicted as the sum of intercept β₀ and the 4 functions; RR₀, RR₋₁, the timing within the ventilation cycle and the timestamp of each beat. The lower panel shows the output whereby an example of the reconstructed signal is shown. The fitted values for PP, based on the unique values of predictors of every beat are projected over the raw signal for comparison. B. Formula for quantification of the effect of ventilation (function in the middle panel) as a percentage of the range of the function over the intercept of the model.

FIG. 3 illustrates pre- and post-leg raising (LR) plots of Ventilation induced Pulse Pressure Variation (%) (VPPV) (A) as determined using embodiments of the present invention and Pulse Pressure Variation (%) (PPV) (B) as measured using prior art techniques, as can be obtained using embodiments of the present invention. Individual values before LR are plotted against their absolute change after the LR manoeuver for VPPV (C) and PPV (D). The Spearman rank correlation coefficient is 0.92 and 0.38 for VPPV and PPV respectively, indicating a strong negative correlation between baseline VPPV and changes in VPPV with leg raising (LR). The shadow of the regression line signifies it's 95% confidence interval.

FIG. 4 illustrates raw data divided in 9 regions using 10 knots (left panel) and individual cubic polynomial fits to the 9 regions without constraints (right panel), as can be used in embodiments of the present invention.

FIG. 5 illustrates 3 examples of splines for the raw data as used in an exemplary embodiment of the present invention.

FIG. 6 and FIG. 7 illustrates experimental results as can be obtained using embodiments according to the present invention.

The drawings are only schematic and are non-limiting. In the drawings, the size of some of the elements may be exaggerated and not drawn on scale for illustrative purposes. Any reference signs in the claims shall not be construed as limiting the scope. In the different drawings, the same reference signs refer to the same or analogous elements.

### DETAILED DESCRIPTION OF ILLUSTRATIVE EMBODIMENTS

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes. The dimensions and the relative dimensions do not correspond to actual reductions to practice of the invention.

Furthermore, the terms first, second and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

Moreover, the terms top, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

It will be clear that in embodiments according to the present invention, where reference is made to continuous blood pressure data, pulse pressure and alike, reference mutatis mutandis is made to continuous stroke volume data, stroke volume and alike. Although the examples are mainly expressed with reference to pulse pressure, the same is applicable to stroke volume. It thereby is to be noted that pulse pressure and stroke volume are related parameters.

Where in embodiments of the present invention reference is made to hemodynamic parameters, reference is made to the group of parameters that express a property of the heart-blood vessel functioning, such as for example the heartbeat, the blood pressure, the flow rate, a pressure measured in the heart-blood vessel system.

Where in embodiments of the present invention reference is made to filling parameters, reference is made to a sub-group of the hemodynamic parameters describing the filling state of the heart-blood vessel system. These filling parameters can be divided into the static filling parameters and the dynamic filling parameters. The static filling parameters are the parameters that are measured at the end of the respiratory cycle, at one specific moment in time. The idea behind it is that the respiratory cycle influences the measurement. Therefore, traditionally, a measurement is done at the end of the respiratory cycle. Examples of static filling parameters are the pressure measured in the right atrium, the pressure in the left atrium, the pressure in the peripheral veins, the volume of the left ventricle before it contracts, etc. The dynamic filling parameters are those parameters for which, rather than measuring them at one moment in time, the change of the parameters is measured upon a standardized change of the filling state. Examples of a standardized change are breathing, lifting the legs of the patient (cfr. passive leg raising test).

Where in embodiments of the present invention reference is made to pulse pressure (PP) reference is made to the difference between the systolic and diastolic blood pressure.

Where in embodiments of the present invention reference is made to pulse pressure variation (PPV), reference is made to the change of the pulse pressure due to ventilation, determined according to principles of the prior art. This parameter is calculated as

$$P P V = \frac{maximal PP - minimal PP}{average PP}$$

Where in embodiments of the present invention reference is made to respiratory induced pulse pressure variation (RPPV), reference is made to a parameter expressing (typically in percentages) respiratory induced changes of the filling parameter as obtained using embodiments of the present invention. The latter typically relates to changes induced by ventilation or changes induced by spontaneous breathing.

Where in embodiments of the present invention reference is made to stroke volume variation (SVV), reference is made to the change in the amount of blood ejected from the left ventricle into the aorta with each heartbeat, determined according to principles of the prior art. Similarly, where in embodiments of the present invention reference is made to respiratory induced stroke volume variation (RSVV) reference is made to a parameter expressing respiratory induced changes of this filling parameter as obtained using embodiments of the present invention. The latter typically relates to changes induced by ventilation or changes induced by spontaneous breathing.

Where in embodiments of the present invention reference is made to a deconvolution, reference may be made to additions, such as for example applying a generalized additive model (GAM), but alternatively also may refer to a model taken not only into account pure addition but also the fact that some submodels may influence each other. As the latter requires a longer observation and an increased difficulty to identify quick changes, a trade of may also be made.

According to a first aspect, the present invention relates to a computer-implemented method for determining a dynamic filling parameter for the heart-vessel system of a patient. The method may be especially applicable during surgery or monitoring of living beings having an irregular heartbeat, such as for example living being having atrial fibrillation, although embodiments are not limited thereto. For example, it is an advantage of embodiments of the present invention that methods and systems are provided that allow to obtain an accurate view on a living being's hemodynamics both for living beings having a regular heartbeat as well as for human beings with an irregular heartbeat. The heartbeat may go from regular to 100% irregular rhythm. The heartbeat may go from sinus rhythm to atrial fibrillation. Where in embodiments reference is made to living beings, this may refer to human being as well as to animals.

According to the present aspect, the method comprises receiving electrocardiogram data for a patient over time, receiving data related to the respiratory cycle of the patient over time and receiving continuous blood pressure data over time or continuous stroke volume data over time. It will be clear that the data are corresponding data, for the same patient and for at least a common time period. The receiving may be receiving the data from a measurement system or from a data memory, as well as directly measuring the data on the living being.

The electrocardiogram data express an electrical activity of the heart. These typically are measured at the skin surface. It may be in one example obtained with a conventional ECG system. The system may be a system having any suitable number of electrodes, such as for example 3, 5, 6, 12 or more electrodes. The electrode configuration used may be any suitable type of configuration. The data may for example be recorded using the lead II, although embodiments are not limited thereto. The data may for example be sampled at a frequency between 100 Hz and 1000 Hz, although embodiments are not limited thereto. Examples of electrical heart activity parameters that can be used are the timing between two R waves, also referred to as RR intervals, although also other parameters can be used.

The data related to the respiratory cycle of the patient over time may comprise a frequency of the respiratory cycle. The latter is especially applicable when mechanical ventilation is applied. In some embodiments, which can be used for patients that are provided with mechanical ventilation, the data may be a frequency of the ventilator. Alternatively or in addition thereto, the data may also be the exact timing of one or more of the phases of the ventilation. Further alternatives are data of the respiratory cycle available from a monitor or based on bio-impedance measurements. For spontaneous breathing patients, the data related to the respiratory cycle of the patient may for example be a timing of one or more phases of the breathing cycle, e.g. with respect to the ECG data or pulse pressure. In some embodiments, the length of the respiratory cycle can be used.

Receiving continuous blood pressure data may in one embodiment be performed by performing invasive arterial blood pressure measurements, although embodiments are not limited thereto. For example in one embodiment, the continuous blood pressure data also could be obtained by measuring continuously blood pressure at the finger of the patient.

The method further comprises correlating said electrocardiogram data, said data related to the respiratory cycle and said continuous blood pressure data or continuous stroke volume data thereby expressing the pulse pressure or stroke volume as a deconvolution of at least a function of the electrocardiogram data and a function of the respiratory cycle data.

In some embodiments, the method comprises expressing the pulse pressure as follows

PP=β₀+ƒ(ECG data)+ƒ(respiratory cycle).

β₀ thereby expresses the average pulse pressure, whereas the functions ƒ(ECG data) and ƒ(respiratory cycle) are functions of the electrical heart activity data and of the respiratory cycle data as mentioned above. In some embodiments, the function of the ECG data may be a function of one or more ECG parameters, such as for example

PP=β₀+ƒ(RR₀,RR₋₁)+ƒ(respiratory cycle).

Whereas RR₀ and RR₋₁ are advantageous to use since these are easy measurable and have a good reproducibility, also other components could be used alternatively or in addition thereto, such as for example Q or S wave components. Nevertheless, the latter are not always easy to measure.

In some embodiments, the function of the ECG data may be a function of the duration of the first preceding RR interval (RR₀) and a function of the duration of the RR interval preceding the RR₀ interval, resulting in:

PP=β₀+ƒ(RR₀)+ƒ(RR₋₁)+ƒ(respiratory cycle).

In some embodiments, additionally also a slow variation over time of the pulse pressure which can be caused by other phenomena can be taken into account. The latter may for example be expressed as f(trending). An example of these slow variations are the Mayer waves. These are a group of slow frequency variations of PP over time, caused by oscillations in baroreceptor and chemoreceptor reflex control systems.

In some embodiments, the method also takes into account a possible small error that can occur. Such an error may be caused by other effects, such as for example measurement errors. Advantageously such an error contribution is limited to e.g. less than 5%, e.g. less than 1%, e.g. less than 0.5%.

Expressing the pulse pressure as a deconvolution of at least a function of the ECG data and a function of the respiratory cycle data may for example comprises expressing the pulse pressure, for each observation period as a gam model.

The latter could for example be expressed as:

PP=β₀+ƒ(RR₀)+ƒ(RR₋₁)+ƒ(respiratory cycle)+ƒ(trending)+ε.

In one example, the functions used may be penalized cubic regression splines for RR₀, RR₋₁ and the time stamp, and a cyclic cubic spline for the respiratory cycle, e.g. timing within the respiratory cycle.

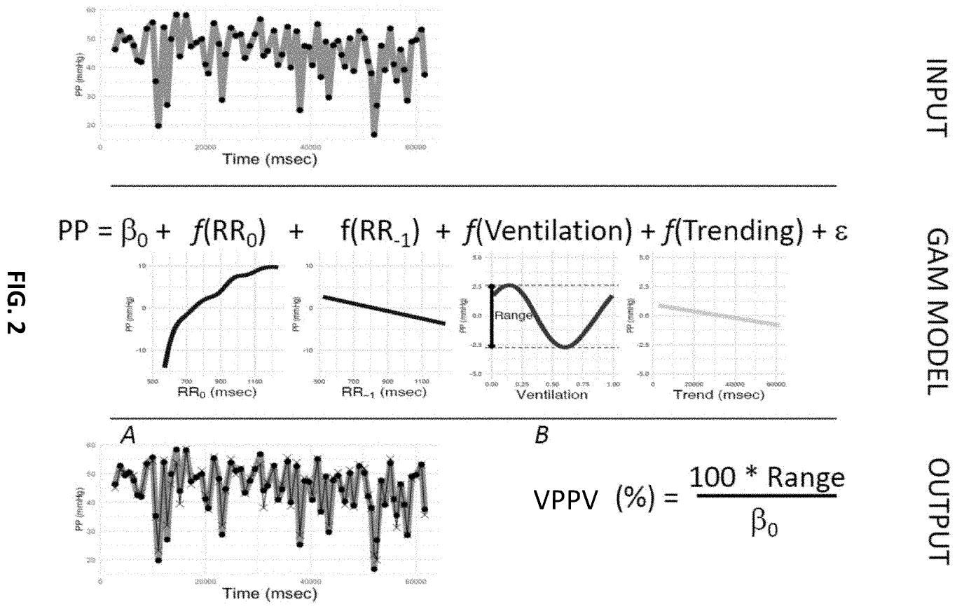

By way of illustration, a schematic representation of the different contributions is shown in FIG. 2 (central drawing).

The method also comprises determining or estimating from the expression of the pulse pressure or stroke volume a dynamic filling parameter representative for the hemodynamics of the patient.

The dynamic filling parameter may be the respiratory induced pulse pressure variation RPPV, the respiratory induced stroke volume variation RSVV, . . . . An example dynamic filling parameter is shown in FIG. 2, bottom drawing, wherein the RPPV is estimated as follows:

RPPV (%)=(100*range)/β₀

Thus, in some embodiments, the coefficient β₀ may be used for indexing the RPPV or VPPV for the blood pressure. β₀ thereby may be considered as an average blood pressure. RPPV or VPPV thus may be scaled using the inverse of β₀.

In some embodiments, the determined or estimated dynamic filling parameter may be used for predicting the fluid responsiveness e.g. to describe the hemodynamic state that administering extra fluids will result in an increased cardiac output. The latter may be used for example to amend the treatment of the patient. For example, if an anesthetist thinks a raise in cardiac output is beneficial for a patient, he/she can use dynamic filling parameters to decide if administering extra fluids is a valid measure. If fluid loading is not an option (if the patient is not fluid responsive), other therapeutic options are to be used (like administering medications like inotropics (e.g. dobutamine, milrinone etc), because administering fluid in this situation will only have detrimental effects for the patient (like peripheral and lung edema formation.

The method may be a computer-implemented method.

It is an advantage of embodiments of the present invention that embodiments make it possible to fully determine the impact of mechanical ventilation on pulse pressure, irrespective of the heart rhythm.

It is also an advantage of embodiments of the present invention that the parameter for hemodynamic filling can be measured continuously, making it clinically more relevant over methods that depend on maneuvers (e.g. like leg up/fluid challenge) or techniques that rely on the unpredictable occurrence of extra-systoles.

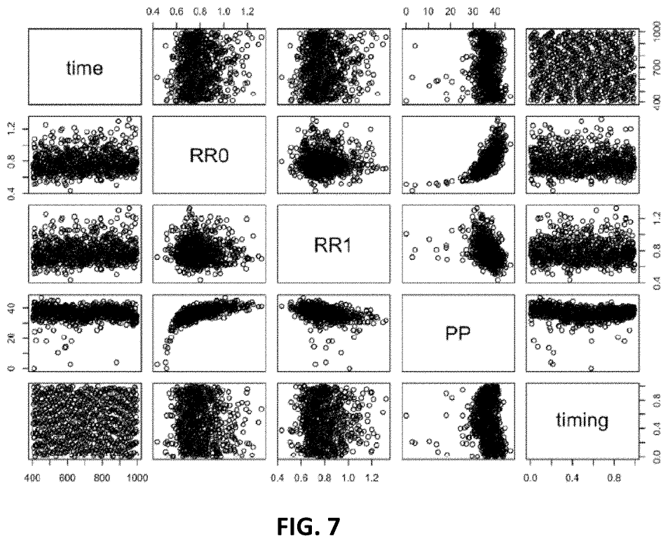

By expressing the pulse pressure as a deconvolution of the different effects, accurate determination of the dynamic filling parameter can be obtained, as is illustrated in FIG. 6 and FIG. 7. FIG. 6 illustrates the component contribution for ventilation (when ventilation is applied, which is not necessarily the case since the idea also works for spontaneous breathing living beings). FIG. 7 illustrates the raw data of the different heart beats of the observation method.

By way of illustration, embodiments of the present invention not being limited thereto, further standard and optional features will be illustrated by way of a study using an exemplary method described below. The example illustrates how cardio-pulmonary interaction can be quantified in patients with atrial fibrillation. It shows how to analyse and identify the individual causes of variation in pulse pressure and therefore allows to quantify the respiratory induced pulse pressure variation, in the present example being a ventilation induced pulse pressure variation (VPPV) in patients with AF. This study illustrates the principle for patients with active AF scheduled for an ablation of the pulmonary vein under general anaesthesia.

The study was done on ten AF patients who were planned for a pulmonary vein isolation under general anesthesia. These patients needed to fulfilled following criteria: (1) Age>18 years, (2) Atrial fibrillation during the study period and (3) ASA 1, 2 or 3. Further exclusion criteria were as follows: (1) Participation in a clinical trial within the past 30 days, (2) Chronic Obstructive Pulmonary Disease, (3) Right ventricular failure, (4) Aortic valve insufficiency or stenosis and (5) an average heart rate of >140 beats/minute.

The procedure followed in the study was as follows: All patients had a standard induction and maintenance of anaesthesia. A combination of bolus sufentanil 0.1-0.2 μg/kg, propofol 2 mg/kg and cisatracurium 0.15 mg/kg were used for induction. After intubation, sevoflurane (End Tidal fraction 1.7-2.0%) was used for maintenance, supplemented with aliquots of 5 μg sufentanil. Besides the standard monitoring (5-lead ECG, pulse oximetry and noninvasive blood pressure), a 3F catheter (Leadercath Arterial, Vygon, France) was placed in the radial artery. The transducer was leveled at the mid-axillary line and zeroed to atmospheric pressure.

During the different registration periods, ECG (II and V2) and arterial pressure signals were simultaneously registered. Each registration channel stored the signals with a sample rate of 1000 Hz using LabSystem Pro v2.4a (BARD® Electrophysiology, Lowell, Mass., USA). Two registration periods were used, with each period lasting 60 seconds: one in baseline conditions with the patient in supine position and one with the legs up. The ventilator settings were the same for both periods: 12*8 ml/kg with a PEEP of 5 cm H₂O.

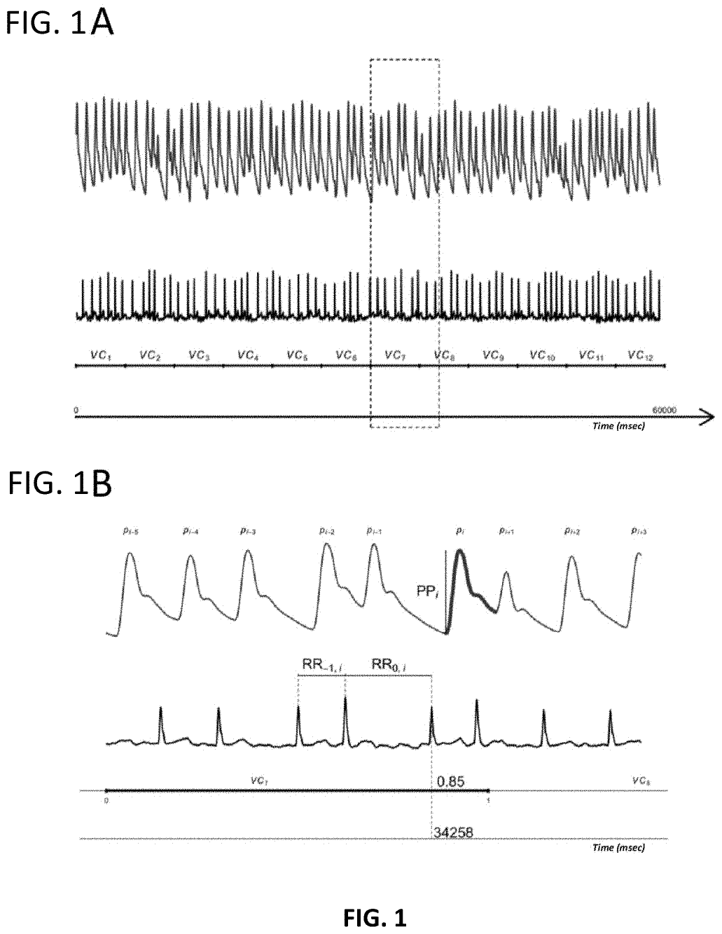

The data analysis was as follows: Data were analyzed off-line using a personal Matlab®-script based on Li et al. as described in “On an automatic delineator for arterial blood pressure waveforms.” Biomedical Signal Processing and Control. 2010; 5:76-81. For each observation period the classic PPV was calculated as previously published by Michard F. in “Changes in arterial pressure during mechanical ventilation. Anesthesiology (2005) 103, 419, for comparison. From the raw data of a 60 sec observation period (FIG. 1 part (A)), 4 variables and the pulse pressure (PP), were determined for every individual beat. The two first variables, the preceding RR-interval (RR₀) and the second preceding RR-interval (RR₋₁) were determined (FIG. 1 part (B)) as previously described by Wyffels et al. in “Dynamic filling parameters in patients with atrial fibrillation: Differentiating Rhythm induced from Ventilation induced variations in Pulse Pressure”, Am J Physiol-Heart C, American Physiological Society (2016) 310.

The method according to the exemplary method of an embodiment of the present invention thus makes use of the factors RR₀ and RR₋₁ being defined as the preceding and pre-preceding RR interval of each individual beat respectively, as illustrated in FIG. 1. For every individual beat the PP increases in a non-linear way with increasing RR₀, as illustrated in FIG. 2. This has been attributed to the difference in filling times of the ventricle. In contrast to RR₀, RR₋₁ has a negative effect on the PP, as illustrated in FIG. 2. The shorter this interval, the higher the resultant PP is. This has been explained by changing contractility, possibly combined with a decrease of LV afterload.

The third variable of the model is the timing of each beat within the respiratory cycle. The timing of the R wave of the ECG was coded as the relative position within its 5 second respiratory cycle, as shown in FIG. 1 part (B), line 3. As fourth variable for trending, the absolute time within the 60 sec observation period was used, as shown in FIG. 1 part (B), line 4.

Starting from the raw pulse pressure data of each observation period of 60 s (FIG. 2 upper panel), the individual impact of each of the individual variables, RR₀, RR₋₁, ventilation and trending was identified. A generalized additive model (gam) was determined to predict the pulse pressure PP based on ‘RR₀’ and ‘RR₋₁’ (the effect of an irregular heartbeat), ‘Ventilation’ (the effect of ventilation on the other hand) and trending of the PP over time (the effect of low-frequency changes in pulse pressure). A generalized additive model is an expansion of a classic multiple linear regression model by allowing a non-linear function for each of the variables, as shown in FIG. 2.

Gam formula: PP=β₀+ƒ(RR₀)+ƒ(RR₋₁)+ƒ(Ventilation)+ƒ(Trend)+ε

The functions used in the model were penalized natural cubic splines for RR₀ and RR₋₁ and cyclic splines for timing, allowing for flexible non-linear modeling (for further explanation see below). The goodness of fit was evaluated with a modified r², that quantified the explained deviations of the PP's by the model.

The ventilation induced pulse pressure variation VPPV was calculated, in analogy of the classical model for PPV, as the range of impact of ventilation on PP, normalized for the intercept of the model.

VPPV=[max(ƒ(Ventilation))−min(ƒ(Ventilation))]/β₀(FIG. 2)

After testing for normality with the Shapiro Wilk test, data are reported as median [IQR] or mean (SD) as appropriate. Comparisons between the 2 measurement periods were performed using a paired t-test or a paired Wilcoxon test for PPV and VPPV values. Correlation was assessed using the Spearman rank correlation coefficient. P values<0.05 were considered statistically significant.

Goodness of fit of each individual gam model was assessed based on the r².

All statistical analyses were done using R (version 3.5.0) base packages and ‘mgcv’ package (1.8-24) for gam.

TABLE 1
Demographic data of included patients.
Data are given median [range].

| Sex, (men/women) | 6/3 | |
|---|---|---|
| Caucasion, (%) | 100 | |
| Age, (yr) | 59 | [55, 78] |
| Weight, (kg) | 95 | [65, 112] |
| Length (cm) | 183 | [160, 185] |
| Cardiovascular comorbidity, (n) | | |
| Hypertension | 6 | |
| Hypercholesterolemia | 1 | |
| Ischemic Heart disease | 1 | |
| Corrected valvular disease | 1 | |
| Corrected congenital heart disease | 1 | |
| Congestive heart failure | 0 | |
| Diabetes/metabolic syndrome (n) | 3 | |
| Stroke/transient ischemic attack (n) | 2 | |
| Medication (n) | | |
| Amiodarone | 2 | |
| Digoxin | 1 | |
| Flecainide | 2 | |
| Beta-Blockers | 5 | |
| Calcium channel blockers | 2 | |
| ACE inhibitor/AII blockers | 2 | |
| Diuretics | 3 | |
| CHADS-VASC2 score | 1.5 | [1, 5] |

The patient characteristics are displayed in table 1.

The obtained results were as follows. As indicated 10 patients were included in the study. Due to a technical problem with the invasive arterial blood pressure measurement, 1 patient had to be excluded.

For all 18 (baseline and legs up in 9 patients) observation periods, an excellent goodness of fit of the model was observed. The median amount of deviation of PP explained by the model, was 91.3% (IQR: 89.2-94.2). This means that more than 90% of the observed PP fluctuations could be predicted by the model.

RR₀ and RR₋₁, the two predictors to describe the effect of atrial fibrillation were statistically significant in all 18 observation periods. Trending, the predictor for overall PP changes during the observation period was significant in 7 of the 18 observation periods. Ventilation was a significant predictor of VPPV) in 7 of the 9 observation periods before leg raising and in 2 on 9 patients after leg raising. The hearth rate is calculated from the median RR interval of each observation period. The pulse pressure (PP) is calculated as the median of the PP of each observation period. The ventilation induced pulse pressure variation (VPPV) decreased significantly after leg raising, while PP increased significantly with this manoeuvre.

TABLE 2
Comparison between pre and post leg raising (LR).

| | Pre LR | | Post LR | | P-value |
|---|---|---|---|---|---|
| Ventilation | 9.9 | [0.1-27.9] | 1.4 | [0, 11.3] | 0.014 |
| induced Pulse | | | | | |
| Pressure | | | | | |
| Variation | | | | | |
| VPPV (%) | | | | | |
| Pulse Pressure | 134 | [14.5-197.87] | 36.8 | [7.6-192.7] | 0.019 |
| Variation | | | | | |
| PPV (%) | | | | | |
| Heart Rate (bpm) | 80 | [73, 91] | 73 | [64, 75] | 0.09 |
| HR (beats/min) | | | | | |
| Pulse Pressure | 33 | [32, 40] | 48 | [42, 52] | 0.027 |
| in mmHg (PP) | | | | | |

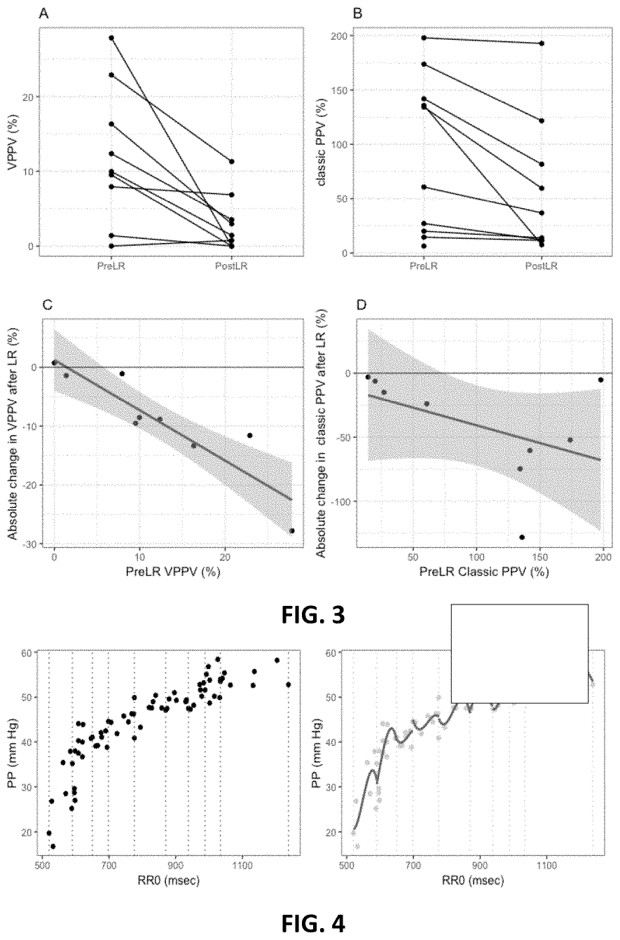

There was a linear relation between the baseline VPPV's and the change in VPPV after LR (p<0.0001). The Spearmans rank correlation coefficient was 0.92 (p=0.0007), as shown in FIG. 3.

PPV values, calculated with the classic formula, were higher than the corresponding VPPV values. PPV before and after the LR differed significantly (table 2). The Spearmans rank correlation coefficient between pre-LR value and its absolute change was 0.38 (p=0.21) as shown in FIG. 3. decreases the impact of mechanical ventilation on the PP, especially when the baseline value is high.

The obtained model is able to retrospectively decompose the successive beat to beat changes in PP, into these 3 sources: intrinsic irregular heart rhythm, mechanical ventilation, and slow PP changes over time. The data show that controlling for irregular heartbeat, more specifically RR₀, is a predictor with the greatest strength and impact of this model. This can be seen from the range of its coefficients and the percentage of explained deviation. This explains why, in contrast to patients with regular heart rhythm, the ventilation induced cyclic changes in PP cannot easily be recognised visually on screen, even when this effect is substantial. As indicated, a generalized additive model (gam) was used. This modelling technique has two advantages. First, it is very flexible. The relationship of each predictor with the dependent variable can be described by splines, a smoothing technique to describe linear or non-linear functions without knowing its exact shape or coefficients, as will be described further.

Second, these relationships were calculated simultaneously and were additive. This means that the model consists of a sum of these individual functions. The function of each predictor is determined independent of each other. Because of these two properties this approach was used to quantify the isolated impact of ventilation. To do this, the classic formula to calculate PPV was slightly changed: The range of changes in PP imposed by the ventilator was divided by the mean value of PP (β0 of the model, FIG. 2).

In the approach according to embodiments of the present invention, a method to filter the whole signal into its different driving processes is illustrated. This enables to quantify the isolated effect of mechanical ventilation on PP. Some of the settings of the model, like epoch and exact timing of the ventilator, were arbitrarily chosen. The model in the example was based on a 60 s window, because this epoch seemed a reasonable period in clinical practice. A shorter epoch would be able to pick up more short-term changes. Calculations based on a wider window on the other hand would provide a more stable but damped model, less prone to measurement error. Future research, based on longitudinal data, is needed to determine the optimal epoch.

The exact timing of the ventilation could not be measured in the protocol. As a result, small shifts of the real to the arbitrarily set respiratory cycle in the current study may have occurred in the analysis. Although it is thought this does not impact the measurement of the range of these cyclic changes, incorporating the exact time-stamped data from the ventilator mechanics into the model may provide an even more accurate physiologic insight into these studied interactions.

The example shows the ability of this algorithm to quantify ventilation induced PPV in patients with AF in the presence of different loading conditions, thereby providing a potential tool for assessing fluid responsiveness in patients with AF. The impact of mechanical ventilation on PP can thus be quantified in patients with AF. Furthermore, the new parameter behaves like classic dynamic filling parameters i.e. PPV.

As indicated above, the individual functions used in the General Additive Model will now further be described. In the example, the individual functions used in the Genera Additive Model are natural cubic splines. This is a specific type of spline. Splines are an elegant method to perform a regression without knowing the exact underlying relation between independent and dependent variables. Hypothetically, this relation can have all forms from linear to higher order polynomials, from exponential to sinusoidal etc. This method has some specific characteristics. Spline regression is a penalized, local, smoothing technique based on a cubic polynomial regression.

The basis for this method is the cubic polynomial:

ƒ(x)=β₀+β₁x+β₂x²+β₃x³

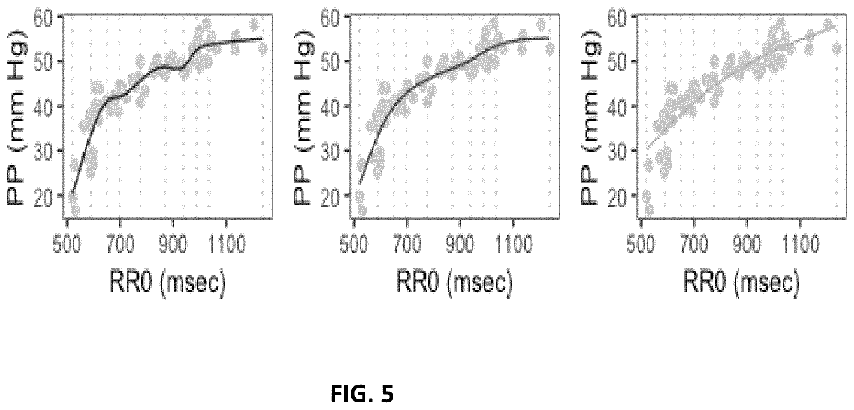

The cubic polynomial formula is not applied to the whole data set, but only to a subset. FIG. 4 shows the individual data points of a 60 s observation period. For simplicity, only the relation between RR₀ and PP is considered. In this example, the whole data is divided into 9 subsets. The exact place of the 10 boundaries (‘knots’) is based on the percentiles of the RR₀ values. Each subset has an equal amount of datapoints. For each subset a cubic polynomial is (locally) applied. So, the formula for a model with k knots can be written as:

$${y}_{i} = \{ \begin{matrix}{β}_{0 , 1} + {β}_{1 , 1} {x}_{i} + {β}_{2 , 1} {x}_{i}^{2} + {β}_{3 , 1} {x}_{i}^{3} , & if {k}_{1} < {x}_{i} < {k}_{2} \\ {β}_{0 , 2} + {β}_{1 , 2} {x}_{i} + {β}_{2 , 2} {x}_{i}^{2} + {β}_{3 , 2} {x}_{i}^{3} , & if {k}_{2} < {x}_{i} < {k}_{3} \\ & … \\ {β}_{0 , k - 1} + {β}_{1 , k - 1} {x}_{i} + {β}_{2 , k - 1} {x}_{i}^{2} + {β}_{3 , k - 1} {x}_{i}^{3} , & if {k}_{k - 1} < {x}_{i} < {k}_{k}\end{matrix} \\ or as : {f}_{j} ( {x}_{i} ) = {β}_{0 , j} + {β}_{1 , j} {x}_{i} + {β}_{2 , j} {x}_{i}^{2} + {β}_{3 , j} {x}_{i}^{3} if {k}_{j} < {x}_{i} < {k}_{j + 1}$$

If no constraints are placed on these 9 different cubic polynomial fits, the resulting graphical display of the model would look like FIG. 4 Right Panel.

There are at least 2 problems with this regression: First, these 9 individual regressions are not continuous. An example of this is the transition at the 4th and 8th knot. There seems to be a ‘jump’ in the regression function at RR₀=698 msec and RR₀=1034 msec. Secondly, in some knots the data seems to be continuous but the regressionline has an overly sharpe edge. This phenomenon can be seen at the 6th knot (RR₀=870 msec). To overcome these problems and optimize the smoothing properties of the model, the following constraints are defined to the individual cubic polynomial fits. At each knot the functions need to be continuous up to the second derivative.

$$\{ \begin{matrix}{f}_{i} ( {k}_{j} ) = {f}_{i + 1} ( {k}_{j} ) \\ {f}_{i}^{′} ( {k}_{j} ) = {f}_{i + 1}^{′} ( {k}_{j} ) \\ {f}_{i}^{″} ( {k}_{j} ) = {f}_{i + 1}^{″} ( {k}_{j} )\end{matrix}$$

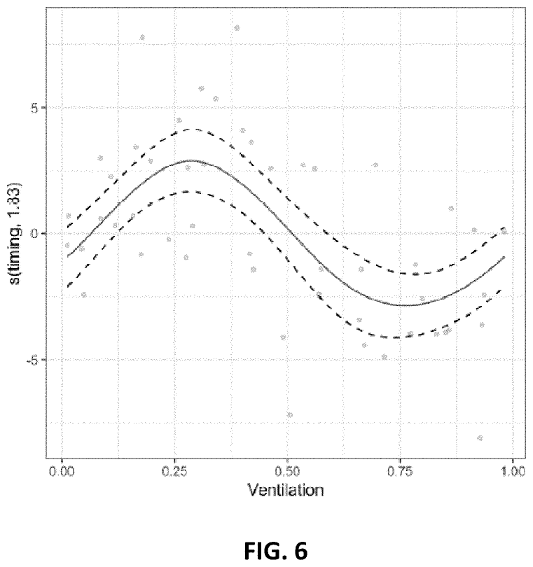

Some examples of such a fit can be seen in FIG. 5.

As can been seen in FIG. 5, there are still multiple solutions to the formula. The minimalization of the following formula is used to choose the optimal fit, to find the optimum between overfitted (graph on the right) and underfitted (graph on the left) models.

$${\sum }_{i = 1}^{n} {( {y}_{i} - f ( {x}_{i} ) )}^{2} + λ \int {f}^{″} ( t ) dt$$

This formula consists of 2 parts. On the left is the classical RSS (Residual Sum of Squares). Minimizing this part of the formula leads to a model that has the least overall prediction error, but has the highest tendency for overfitting. The right part of the formula measures for the impact of the higher-order coefficients (second derivative), and counterbalances this tendency. λ is a penalty factor. Choosing a low λ yields a model that is allowed to be ‘wiggly’. Higher λ's shifts the model to less flexible versions, ultimately leading to a linear function. There are different ways of determining the optimal λ. In our analysis we used the REML (Restricted Maximum Likelihood) approach.

In a second aspect, the present invention relates to a system for predicting a dynamic filling parameter for the heart-vessel system of a patient. The system may be especially suitable for performing a method according to the aspect as described above, although embodiments are not limited thereto. The system comprises an electrocardiogram data receiving means for receiving electrocardiogram data for a patient over time and a respiratory cycle data receiving means for receiving data related to the respiratory cycle of the patient over time. The system also comprises a continuous blood pressure data receiving means for receiving continuous blood pressure data for a patient over time or a continuous stroke volume data receiving means for receiving continuous stroke volume data for a patient over time. The electrocardiogram data receiving means may be an input port for receiving data from an electrocardiogram recording device or from a memory. The electrocardiogram data may be data corresponding with a full electrocardiogram signal, but may also comprise only particular details thereof, such as for example the duration of the most recent RR interval (RR₀), the duration of the RR interval (RR₋₁) preceding the most recent RR interval, with respect to individual heartbeats, etc. Alternatively, the electrocardiogram data receiving means may be the electrocardiogram recording device itself. The respiratory cycle data receiving means may be an input port for receiving data regarding the respiratory cycle. The data may for example be obtained from a mechanical ventilator, from a respiratory monitor, from a bio-impedance measurement device, etc. As indicated above, the data may for example be a ventilation frequency, e.g. when ventilation is applied, or it may for example be a timing of one or more phases of the breathing cycle.

The electrocardiogram data, the data related to the respiratory cycle and the continuous blood pressure data, are corresponding data regarding the patient recorded during a same moment in time.

The system furthermore comprises a processor being configured or programmed for correlating said electrocardiogram data, said data related to the respiratory cycle and said continuous blood pressure data or continuous stroke volume data thereby expressing the pulse pressure or stroke volume as a deconvolution of at least a function of said electrocardiogram data and a function of said respiratory cycle data, and for determining from the expression a value for the dynamic filling parameter representative for the hemodynamics of the patient based on the expression for the pulse pressure or stroke volume.

Further features of the system may be components performing the functionality of method steps or part thereof of methods described in the first aspect. The system may be implemented in software as well as in hardware. Advantageously, the system is programmed for performing the steps of the method for predicting a dynamic filling parameter as described in the first aspect.

According to some embodiments, the system may be a mechanical ventilator wherein the data receiving means and the processor as described above are integrated in the mechanical ventilator.

In still another aspect, the above described system embodiments may correspond with an implementation of the method for predicting a dynamic filling parameter, as a computer implemented invention in a processor. Such a system or processor—the processor also being discussed in functionality in an aspect described above—includes at least one programmable computing component coupled to a memory subsystem that includes at least one form of memory, e.g., RAM, ROM, and so forth. It is to be noted that the computing component or computing components may be a general purpose, or a special purpose computing component, and may be for inclusion in a device, e.g., a chip that has other components that perform other functions. Thus, one or more aspects of the present invention can be implemented in digital electronic circuitry, or in computer hardware, firmware, software, or in combinations of them. While a processor as such is prior art, a system or processor that includes the instructions to implement aspects of the methods is not prior art. The present invention thus also includes a computer program product which provides the functionality of any or part of the methods for predicting a hemodynamic filling parameter according to the present invention when executed on a computing device.

In another aspect, the present invention relates to a data carrier, e.g. a non-transitory data carrier, for carrying such a computer program product. Such a data carrier may comprise a computer program product tangibly embodied thereon and may carry machine-readable code for execution by a programmable processor. The present invention thus relates to a carrier medium carrying a computer program product that, when executed on computing means, provides instructions for executing any of the methods as described above. The term “carrier medium” refers to any medium that participates in providing instructions to a processor for execution. Such a medium may take many forms, including but not limited to, non-volatile media, and transmission media. Non-volatile media includes, for example, optical or magnetic disks, such as a storage device which is part of mass storage. Common forms of computer readable media include, a CD-ROM, a DVD, a flexible disk or floppy disk, a tape, a memory chip or cartridge or any other medium from which a computer can read. Various forms of computer readable media may be involved in carrying one or more sequences of one or more instructions to a processor for execution. The computer program product can also be transmitted via a carrier wave in a network, such as a LAN, a WAN or the Internet. Transmission media can take the form of acoustic or light waves, such as those generated during radio wave and infrared data communications. Transmission media include coaxial cables, copper wire and fibre optics, including the wires that comprise a bus within a computer.

1.-18. (canceled)

19. A computer-implemented method for predicting a dynamic filling parameter for a heart-vessel system of a patient, the method comprising:
receiving electrocardiogram data for the patient over time,
receiving data related to a respiratory cycle of the patient over time,
receiving continuous blood pressure data respectively continuous stroke volume data of the patient over time,
said electrocardiogram data, said data related to the respiratory cycle and said continuous blood pressure data respectively continuous stroke volume data being corresponding data regarding the patient recorded during a same moment in time,
the method further comprising
correlating said electrocardiogram data, said data related to the respiratory cycle and said continuous blood pressure data respectively continuous stroke volume data thereby expressing the pulse pressure respectively stroke volume as a deconvolution of at least a function of the electrocardiogram data and a function of the respiratory cycle data, and
determining a value for a dynamic filling parameter representative for the hemodynamics of the patient based on the expression for the pulse pressure respectively stroke volume.

20. A method according to claim 19, wherein expressing the pulse pressure respectively stroke volume as a deconvolution of at least a function of said electrocardiogram data and a function of said respiratory cycle data comprises applying an additive model for the pulse pressure respectively stroke volume as function of at least said electrocardiogram data and said respiratory cycle data.

21. The method according to claim 20, wherein the additive model is a gam model.

22. The method according to claim 19, wherein the dynamic filling parameter representative for the hemodynamics of the patient is an expression for the variation of the pulse pressure induced by the respiratory cycle.

23. The method according to claim 19, wherein expressing the pulse pressure respectively stroke volume as a deconvolution of at least a function of said electrocardiogram data comprises expressing the pulse pressure respectively stroke volume as a deconvolution of at least a function of the duration of a preceding RR interval in an ECG wave,
wherein the RR intervals are calculated for every individual heartbeat considered.

24. The method according to claim 22, wherein expressing the pulse pressure respectively stroke volume as a deconvolution of at least a function of said electrocardiogram data comprises expressing the pulse pressure respectively stroke volume as a deconvolution of at least a function of the duration of the most recent RR interval (RR0) and a function of the duration of the RR interval (RR−1) preceding the most recent RR interval,
wherein the RR intervals are calculated for every individual heartbeat considered.

25. The method according to claim 19, wherein expressing the pulse pressure respectively stroke volume as a deconvolution of at least a function of said electrocardiogram data comprises applying a general additive model for the pulse pressure respectively stroke volume as function of at least a function of the duration of the most recent RR interval (RR0) and a function of the duration of the RR interval (RR−1) preceding the most recent RR interval,
wherein the RR intervals are calculated for every individual heartbeat considered.

26. The method according to claim 19, wherein the expression of the pulse pressure respectively stroke volume furthermore comprises a component 130 expressing the average pulse pressure.

27. The method according to claim 23, wherein the function of the duration of a preceding RR interval is a spline.

28. The method according to claim 19, wherein expressing the pulse pressure respectively stroke volume as a deconvolution of at least a function of the respiratory cycle data comprises expressing the pulse pressure respectively stroke volume as a deconvolution of at least a function of the timing of each heartbeat with the respiratory cycle.

29. The method according to claim 27, wherein said function of the timing of each heartbeat with the respiratory cycle is a spline.

30. The method according to claim 19, wherein the pulse pressure respectively stroke volume is expressed as a deconvolution of at least a function of said electrocardiogram data, a function of said breathing data, and an additional function expressing a slow variation of the pulse pressure.

31. The method according to claim 19, wherein the respiratory cycle data are ventilation data.

32. The method according to claim 19, the method being implemented as a computer program software.

33. A system for predicting a dynamic filling parameter for the heart-vessel system of a patient, the system comprising:
an electrocardiogram data receiver configured for receiving electrocardiogram data for a patient over time,
a respiratory cycle data receiver configured for receiving data related to the respiratory cycle of the patient over time,
a continuous blood pressure data receiver configured for receiving continuous blood pressure data for a patient over time or a continuous stroke volume data receiving means for receiving continuous stroke volume data for a patient over time respectively,
said electrocardiogram data, said data related to the respiratory cycle and said continuous blood pressure data respectively continuous stroke volume data being corresponding data regarding the patient recorded during a same moment in time, and
a processor, the processor being configured for correlating said electrocardiogram data, said data related to the respiratory cycle and said continuous blood pressure data thereby expressing the pulse pressure respectively stroke volume as a deconvolution of at least a function of electrocardiogram parameters and a function of respiratory cycle parameters, and for determining a value for a dynamic filling parameter representative for the hemodynamics of the patient based on the expression for the pulse pressure respectively stroke volume.

34. The system according to claim 33, wherein the system furthermore is programmed for performing a method comprising the steps of:
receiving electrocardiogram data for the patient over time,
receiving data related to a respiratory cycle of the patient over time,
receiving continuous blood pressure data respectively continuous stroke volume data of the patient over time,
said electrocardiogram data, said data related to the respiratory cycle and said continuous blood pressure data respectively continuous stroke volume data being corresponding data regarding the patient recorded during a same moment in time,
the method further comprising
correlating said electrocardiogram data, said data related to the respiratory cycle and said continuous blood pressure data respectively continuous stroke volume data thereby expressing the pulse pressure respectively stroke volume as a deconvolution of at least a function of the electrocardiogram data and a function of the respiratory cycle data, and
determining a value for a dynamic filling parameter representative for the hemodynamics of the patient based on the expression for the pulse pressure respectively stroke volume.

35. The system according to claim 33, wherein the electrocardiogram data receiver is an ECG monitor.

36. The system according to claim 33, wherein the respiratory cycle data receiver is a ventilator or a bio-impedance measuring device.
